(19) United States
(12) Patent Application Publication
(10) Pub. No.: US 2022/0096008 A1
(43) Pub. Date: Mar. 31, 2022

(54) SYSTEM AND METHOD OF SMART HEALTH MONITORING

(71) Applicants: Shiwei Liu, Lehi, UT (US); Tianye Wang, Halifax (CA); Qiaowei Liu, Lianyungang (CN)

(72) Inventors: Shiwei Liu, Lehi, UT (US); Tianye Wang, Halifax (CA); Qiaowei Liu, Lianyungang (CN)

(21) Appl. No.: 17/474,190

(22) Filed: Sep. 14, 2021

Related U.S. Application Data
(60) Provisional application No. 63/085,897, filed on Sep. 30, 2020.

(51) Int. Cl. A61B 5/00; A61B 5/024; A61B 5/021; A61B 5/08; A61B 5/01; A61B 5/145

(52) U.S. Cl. CPC A61B 5/6801; A61B 5/02416; A61B 5/742; A61B 5/0004; A61B 5/7264; A61B 5/021; A61B 2503/40; A61B 5/01; A61B 5/14542; A61B 5/0022; A61B 5/746; A61B 2560/0214; A61B 5/0816

(57) ABSTRACT

A system and method of smart health monitoring comprises an attachable device having a microcontroller and circuitry, and an external processing device, for effectively predicting the vital health signs of the aquatic animals. The attachable device employs an imaging device and a plurality of sensors to collect the plurality of vital health signs of the aquatic animal. The attachable device is attached on a targeted area of the aquatic animal under measurement. The smart health monitoring system and method of the present invention utilizes the machine learning models to generate a medical model of the individual aquatic animal to obtain more accurate vital health signals.

### CROSS-REFERENCE TO RELATED APPLICATIONS

This application claims the benefit of U.S. Provisional Application No. 63/085,897, filed on Sep. 30, 2020.

### BACKGROUND OF THE INVENTION

### Field of the Art

The present invention relates to a field of smart health monitoring. More particularly, the present invention relates to a system and method of smart health monitoring for aquatic animals, which implements Internet of Things technologies and machine leaning techniques, to infer vital health signals more accurately.

### Discussion of the State of the Art

Health and wellness are of utmost importance for any living being, be it human or animal, and so monitoring it on regular basis is a necessity. With the advancement in technology, the health monitoring systems have coordinately evolved over the years, with a sole aim of assisting people to actively monitor their health status with respect to fitness level as well as medical level.

The measurement of vital signs is critical to the treatment and management of many medical conditions for a variety of target individuals or populations. Some of the prior arts have been described hereunder which disclose about the health monitoring devices, systems and methods.

The prior art WO2021046237A1 discloses a wearable biometric sensor technology for physiological monitoring for medical, health, and fitness applications. Further it discloses a biometric waveform analysis system utilized to generate a physiological assessment using a wearable device having a sensor system, metric output generator, waveform analysis, and control processor configured to control the sensor system, the metric output generator, and the waveform analysis engine.

Another prior art U.S. Ser. No. 10/973,422B2 discloses a device and method for non-invasively measuring arterial stiffness using pulse wave analysis of photoplethysmogram data. The wearable biometric monitoring devices for measuring arterial stiffness have the ability to automatically and intelligently obtain PPG data under suitable conditions while the user is engaged in activities or exercises, providing good-quality PPG data for PWA while conserving power of the wearable biometric monitoring devices.

Yet another prior art US20210110927A1 discloses a method of extracting a respiratory rate from a photoplethysmogram (PPG) using a machine learning model, wherein an artificial neural network model can be trained to predict the respiratory rate using a training dataset containing PPG data. The artificial neural network model can include a series of convolutional layers used to identify a PPG signal in the PPG data and remove artifacts contained in the PPG data, a fast Fourier transform (FFT) layer used to identify PPG frequencies in the PPG data, and a dense layer used to decode a respiratory rate value from the PPG frequencies.

Still another prior art U.S. Ser. No. 10/433,726B2 discloses health monitoring systems, methods, and devices to remotely monitor and track patient health status. The health-monitoring system comprising IoT-vitals sensing nodes joined to a patient's body, sensing vital characteristics, employing wireless transmission circuitry transmitting sensed data by a short-range network, a local gateway having wireless circuitry receiving transmitted data from the IoT-vitals sensors, software (SW) executing on a processor from a non-transitory medium, the SW processing the transmitted data received, and transmission circuitry transmitting processed data over a long-range network.

Further prior art US20210022660A1 discloses systems and methods for collecting and analyzing vital sign information to predict a likelihood of a subject having a disease or disorder, wherein the system for monitoring a subject comprise sensors which are configured to acquire health data comprising vital sign measurements of the subject over a period of time, and a mobile electronic device to process the health data and provide output.

Yet further prior art AU2019101396A4 is disclosed which describes about a smart personal monitoring system to be worn by a first user, comprising an accelerometer to continuously detect physical movements comprising falls of the first user, and a photoplethysmography (PPG) sensor to continuously measure biometric data. Also, a live messaging module is provided to send notification message to one or more second users associated with the first user through a network when event associated with the first user occurs.

Still further prior art U.S. Ser. No. 10/722,749B2 is disclosed which describes about a predictive health monitoring using neural networks, comprising a wearable device with biometric sensors, a database containing data from multiple users across many categories of health-related factors, a first set of neural networks trained on the database that makes health predictions based on a single health factor, and a second neural network that makes health predictions based on a combination of the predictions made by the first set of neural networks.

Although devices, systems and methods for health monitoring have been described by the abovementioned prior arts, they are largely limited to only human health and wellness. Therefore, there remains a significant need and a void in the market to provide a smart health monitoring system and method for aquatic or marine animals. Because, by keeping aquatic animals healthy, the livelihoods of millions around the world can be secured, the diversity of life below water can be protected, and food security can be ensured for our future generations.

Aquatic animals can also be affected by infectious diseases just like terrestrial animals and humans, which may be caused by viruses, bacteria, fungi, protozoa and parasites. Hence, there exists a need to provide a smart wearable health monitoring system and method to effectively monitor health of aquatic animal to protect the sustainability of commercial and recreational fisheries, and the productivity of aquaculture industries.

Moreover, the conventional smart health monitoring systems do not teach about monitoring the vital signs by reconstructing an accurate 4-dimensional representation of the micro blood vessels in the targeted area of the human body, nor are they specifically tailored towards generating a personalized baseline profile. However, the smart health monitoring system of the present invention is applicable to human being with a customized hardware design. Therefore, the unique characteristics and features of the present invention are unrepresented within the conventional arts.

The applicant is unaware of the existence of any such system and method to monitor health of the aquatic animals that contains the abovementioned features and addresses the above described shortcomings in the prior arts. More specifically, there is no such IoT-based system and method known in the prior art that is capable of accurately and frequently measuring vital signs of the aquatic animals, wherein the vital signs include body temperature, blood pressure, peripheral capillary oxygen saturation (SpO2), pulse rate, and respiration rate. Furthermore, no such system exists which uses a data visualization platform and automated machine learning platform, to quickly analyze the health-related data from the aquatic animals and generate a personalized baseline profile. Also, no existing system provides an extraction of more detailed vital signs from the 4D micro blood vessel time-series data captured via various sensors to reconstruct aquatic animal's hemodynamics system.

### SUMMARY OF THE INVENTION

Accordingly, the inventor has conceived and reduced to practice, in a preferred embodiment of the invention, a smart health monitoring system and method which is capable of accurately as well as frequently measuring a plurality of vital health signs including, but not limited to, body temperature, blood pressure, peripheral capillary oxygen saturation (SpO2), pulse rate, and respiration rate.

An objective of the present invention is to provide a system and method of smart health monitoring which measures the vital health signs of the aquatic animals, in a precise manner.

Another objective of the present invention is to provide a system and method of smart health monitoring which employs Internet of things technologies.

Still another objective of the present invention is to provide a system and method of smart health monitoring which utilizes machine learning models to predict the vital health signs.

Still another objective of the present inventions is to provide a system and method of smart health monitoring which utilizes a data visualization platform to analyze the health-related data and generates a personalized baseline profile.

Also, one more objective of the present invention is to provide a system and method of smart health monitoring which reconstructs the aquatic animal's hemodynamics system.

Another objective of the present invention is to provide a system and method of smart health monitoring which secures livelihoods of millions around the world, protects the diversity of life below water, and ensures the food security for the future generations.

Still another objective of the present invention is to provide a system and method of smart health monitoring which protects the sustainability of commercial and recreational fisheries, and the productivity of aquaculture industries.

The present invention therefore introduces a novel IoT-based system and method of smart health monitoring which comprises an attachable device having a microcontroller and circuitry, and an external processing device, for effectively predicting the vital health signs of the aquatic animals. The attachable device employs an imaging device and a plurality of sensors to collect the plurality of vital health signs of the aquatic animal. The attachable device is attached on a targeted area of the aquatic animal under measurement. The attachable device can be customized in design to be applicable on any aquatic animal depending on size and shape of the aquatic animal.

Additionally, the smart health monitoring system and method of the present invention utilizes the machine learning models to generate a medical model of the individual aquatic animal to obtain more accurate vital health signals. Furthermore, the smart health monitoring system also tracks fluctuations in the vital health signal data.

### BRIEF DESCRIPTION OF THE DRAWINGS

The present invention is illustrated and described herein with reference to the various drawings, in which like reference numbers denote the various components and/or steps, as appropriate. The drawings illustrate example embodiments of the present disclosure and cannot be considered as limiting its scope.

FIG. 1 is a block diagram of a smart health monitoring system, according to an embodiment of the present invention.

FIG. 2 is a block diagram of an attachable device of a smart health monitoring system, according to an embodiment of the present invention.

FIG. 3 is a block diagram of a power module of an attachable device, according to an embodiment of the present invention.

FIG. 4 is a flow diagram that illustrates a method of obtaining a plurality of vital health signals more accurately by implementing a smart health monitoring system, according to an embodiment of the present invention.

FIG. 5 is a flow diagram that illustrates an example method for obtaining a vital health signal prediction from a machine learning model implemented in a smart health monitoring system, according to an embodiment of the present invention.

The figures are described in greater detail in the next section of the patent specification.

### DETAILED DESCRIPTION OF THE INVENTION

The foregoing disclosure has broadly outlined the features and technical advantages of the present disclosure in order that the description of the disclosure that follows may be better understood. It should be appreciated by those skilled in the art that the conception and specific embodiment disclosed may be readily utilized as a basis for modifying or designing other structures for carrying out the same purposes of the present disclosure. The novel features which are believed to be characteristic of the disclosure, both as to its organization and method of operation, together with further objects and advantages will be better understood from the following description. The specification concludes with claims defining the features of the systems and methods that are regarded as novel, it is believed that the systems and methods will be better understood from a consideration of the following description in conjunction with the drawing figures, in which like reference numerals are carried forward.

Before the systems and methods are disclosed and described, it is to be understood that the terminology used herein is for the purpose of describing particular embodiments only and is not intended to be limiting. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element. The terms “including” and/or “having,” as used herein, are defined as comprising (i.e., open language). The terms “a” or “an”, as used herein, are defined as one or more than one. The term “plurality,” as used herein, is defined as two or more than two. The term “another,” as used herein, is defined as at least a second or more. The description may use the terms “embodiment” or “embodiments,” which may each refer to one or more of the same or different embodiments.

For the purposes of the description, a phrase in the form “A/B” or in the form “A and/or B” or in the form “at least one of A and B” means (A), (B), or (A and B), where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables, for example, a phrase in the form “at least one of A, B, and C” means (A), (B), (C), (A and B), (A and C), (B and C), or (A, B and C).

Herein various embodiments of the systems and methods are described. In many of the different embodiments, features are similar. Therefore, to avoid redundancy, repetitive description of these similar features may not be made in some circumstances. It shall be understood, however, that description of a first-appearing feature applies to the later described similar feature and each respective description, therefore, is to be incorporated therein without such repetition.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an embodiment with several components in communication with each other does not imply that all such components are required. To the contrary, a variety of optional components may be described to illustrate a wide variety of possible embodiments of the inventions and in order to more fully illustrate one or more aspects of the inventions. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step).

As used herein the following terms have the meaning given below:

“Aquatic animal”—means, in a preferred embodiment, an animal, either vertebrate or invertebrate, which lives in the water for most or all of its lifetime.

“Targeted area”—means, in a preferred embodiment, a specific body area of an aquatic animal where the measurement is needed.

“Microcontroller”—means, in a preferred embodiment, a compact integrated circuit that incorporates a central processing unit, a memory, and input/output functions.

According to the present invention, the measurement of vital signs is critical for the treatment and management of many medical conditions for a variety of aquatic animals. A smart health monitoring system and method, which is capable of accurately as well as frequently measuring the vital signs including, but not limited to, body temperature, blood pressure, peripheral capillary oxygen saturation (SpO2), pulse rate, and respiration rate is described in the following detailed description of the present invention.

Reference will now be made in detail to a presently preferred embodiment of the invention, an example of which is illustrated in the accompanying drawings.

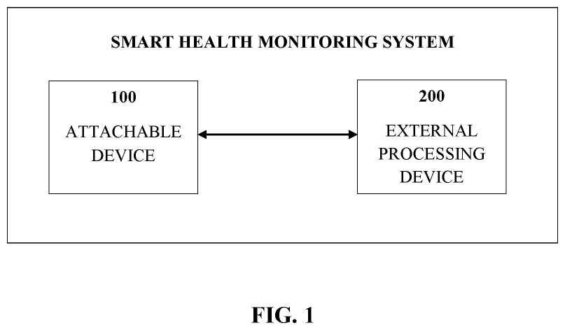

According to an exemplary embodiment of the present invention, a block diagram of an IoT-based smart health monitoring system is shown in FIG. 1. Referring now to FIG. 1, the IoT-based smart health monitoring system comprising: an attachable device 100 configured to be attached on a targeted area of the aquatic animal, to collect various health data of the aquatic animal, and an external processing device 200 configured for machine learning based processing to infer the vital health signals in real time by implementing a machine learning model.

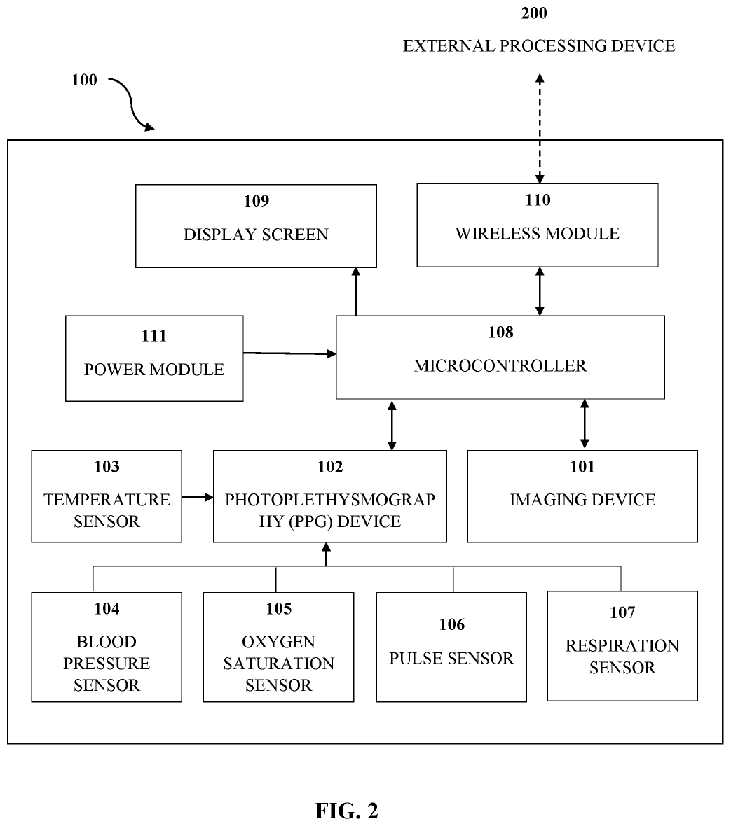

According to another exemplary embodiment of the present invention, various functional components of the attachable device 100 are shown in FIG. 2. Referring to FIG. 2, the attachable device 100 comprising: an imaging device 101 configured to scan the targeted area in order to reconstruct an accurate 4-dimensional representation of a plurality of micro blood vessels present in the targeted area; a photoplethysmography (PPG) device 102 configured to generate a PPG data from a plurality of sensors i.e. a temperature sensor 103, a blood pressure sensor 104, an oxygen saturation (SpO2) sensor 105, a pulse sensor 106, and a respiration sensor 107; a microcontroller 108 with SPI and I2C, programmed to collect, process, and classify the data received from the imaging device 101 and the PPG device 102; a display screen 109 configured to live display a processed data received from the microcontroller 108; a wireless module 110 configured to transmit the processed data wirelessly to the external processing device 200 for machine learning based data processing; and a power module 111 configured to supply power to the microcontroller 108.

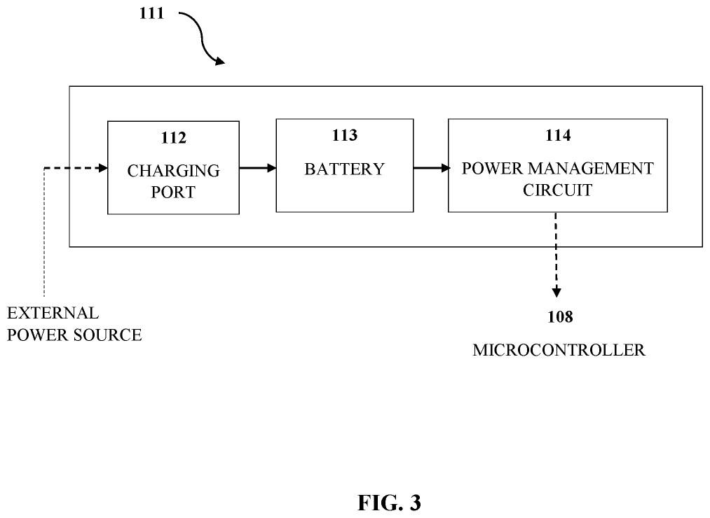

According to another exemplary embodiment of the present invention, a block diagram of various internal components of the power module 111 is shown in FIG. 3. Referring to FIG. 3, the power module 111 comprising: a charging port 112, a battery 113, and a power management circuit 114, wherein the battery 113 is connected to the power management circuit 114 which feeds power to the microcontroller 108, and the battery 113 is charged via the charging port 112 which receives the power when connected to an external charging station (not shown).

According to the abovementioned embodiments of the present invention, the attachable device 100 of the smart health monitoring system is attached on the targeted area of the aquatic animal, wherein the imaging device 101 initially scans the targeted area in order to reconstruct the accurate 4-dimensional representation of the plurality of micro blood vessels present in the targeted area, and the PPG device 102 generates an initial PPG data by the plurality of sensors 103, 104, 105, 106, 107 which detect various body parameters and relay the detected data to the PPG device 102. Further, the initial scan data and the initial PPG data are fed to the microcontroller 108 for automated data collecting, data processing and data analyzing. The processed scan data and PPG data is then live displayed on the display screen 109, which is also transmitted to and stored on the external processing device 200 via the wireless module 110, for machine learning based processing. The processed scan data and PPG data stored on the external processing device 200 hereinafter referred as “recorded data”. Further, the PPG device 102 keeps generating the PPG data in real-time, which is then displayed on the display screen 109 in real-time after processed by the microcontroller 108. The processed PPG data hereinafter referred as “live PPG data”. In the external processing device 200 using a data visualization platform, the scan data from the recorded data is used to reconstruct the detailed 4-dimensional micro blood vessels image, which is further fused with the PPG data from the recorded data, in order to generate a specific baseline profile of the vital health signal data for each aquatic animal under measurement or a specific species of the aquatic animal.

Moreover, the smart health monitoring system implements the machine learning model on the recorded data to generate a medical model of the individual aquatic animal to obtain more accurate vital health signals. For instance, a quantized machine learning model runs on the recorded data to process the recorded data in real time to infer various measurements including, but not limited to pulse rate, blood pressure, SpO2, and respiration rate. The smart health monitoring system utilizes a Deep Neural Network (DNN) model to generate a correlation function that converts the recorded data in real time to identify key features including, but not limited to systolic blood pressure (SBP), diastolic blood pressure (DBP), SpO2, and respiration rate. The smart health monitoring system further utilizes a Recurrent Neural Network (RNN) model to extract more detailed vital signs from the 4D micro blood vessel time-series data captured with the imaging device 101 to reconstruct aquatic animal's hemodynamics system.

According to an embodiment of the present invention, the smart health monitoring system also tracks fluctuations in the vital health signal data through the PPG device 102. In addition, the present invention utilizes the machine learning models to infer if variations from the baseline health signal data are significant in both, aquatic animal under measurement and population.

According to an embodiment of the present invention, the external processing device 200 can be selected from, but not limited to a personal computer, a laptop, a tablet, a smartphone, a mobile phone, and a personal digital assistance. Furthermore, the wireless module 111 transmits the processed scan data and PPG data via a wireless network selected from, but not limited to Wi-Fi, Bluetooth™, ZigBee™, Cellular, and Satellite.

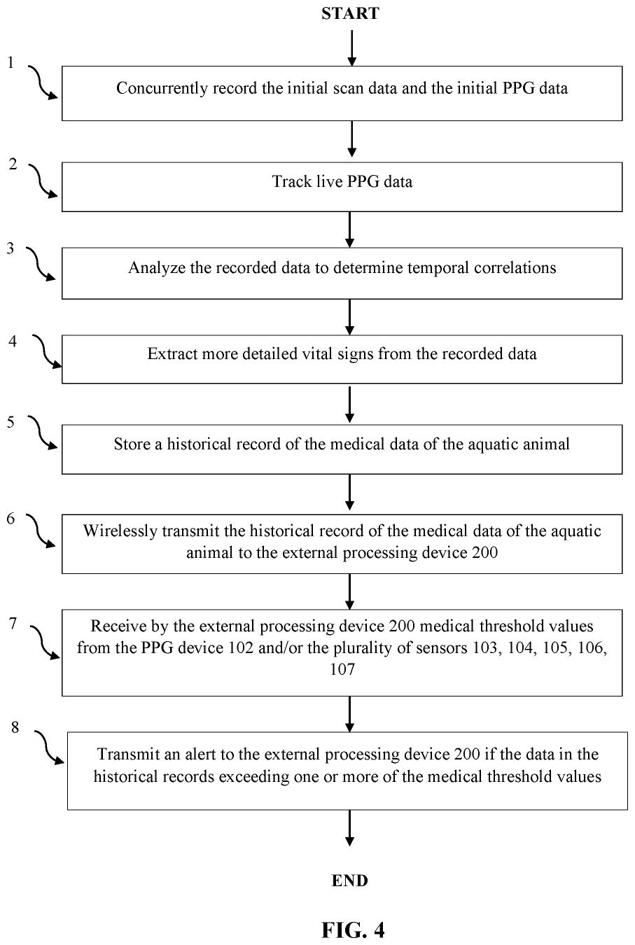

According to another exemplary embodiment of the present invention, the smart health monitoring method is described in the form of a plurality of sequential steps represented by a flow diagram of the present invention as shown in FIG. 4. Referring to FIG. 4, at step 1 in starting with the process, the initial scan data and the initial PPG data is recorded concurrently from the imagine device 101 and the PPG device 102, respectively, and stored on the external processing device 200, wherein the recording of the data lasts for at least 5 minutes.

At step 2, the live PPG data is tracked from the PPG device 102 that is still attached to the aquatic animal.

At step 3, the recorded data is analyzed to determine temporal correlations between the recorded data received from the imagine device 101 and the PPG device 102, and the live PPG data generated by the PPG device 102; wherein the temporal correlations correlate the recorded data to the live PPG data by running the Deep Neural Network (DNN) model to identify key features including, but not limited to systolic blood pressure (SBP), diastolic blood pressure (DBP), SpO2, and respiration rate.

Further at step 4, the smart health monitoring system utilizes a Recurrent Neural Network (RNN) model to extract more detailed vital signs from the recorded data to reconstruct aquatic animal's hemodynamics system.

At step 5, a historical record of the medical data of the aquatic animal is stored on a memory device of the attachable device 100.

At step 6, the historical record of the medical data of the aquatic animal is transmitted wirelessly to the external processing device 200.

At step 7, the external processing device 200 receives one or more medical threshold values from the PPG device 102 and/or the plurality of sensors 103, 104, 105, 106, 107.

Finally at step 8, an alert is being transmitted to the external processing device 200 in response to the data in the historical records exceeding one or more of the medical threshold values.

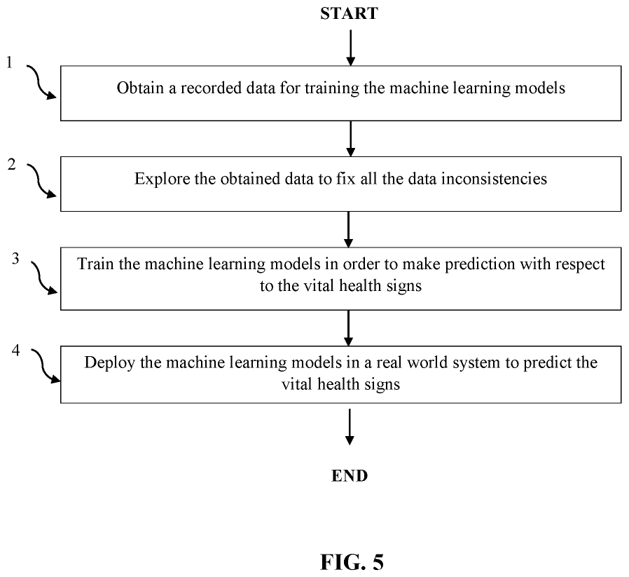

According to an embodiment of the present invention, the smart health monitoring method obtains the vital health signal prediction by implementing the machine learning model, wherein a plurality of sequential steps carried out by the machine learning model using a machine learning algorithm are described in a flow diagram of the present invention as shown in FIG. 5. Referring to FIG. 5, at step 1 in starting with the process, the recorded data including the initial scan data and the initial PPG data is collected to predict the vital health signs.

At step 2, the collected data is further explored to fix all the data inconsistencies by data cleaning before training the machine learning models on it.

At step 3, the machine learning models are trained on the collected data in order to make prediction with respect to the vital health signs. The machine learning models comprising the Deep Neural Network (DNN) model and the Recurrent Neural Network (RNN) model.

At step 4, after training the machine learning models, the machine learning models are deployed in a real world system to predict the vital health signs. For instance, the smart health monitoring system of the present invention. After deploying the machine learning models to the smart health monitoring system, the initial scan data and the initial PPG data generated by the imaging device 101 and the PPG device 102, respectively can be input to the machine learning models, and the machine learning models can analyze the PPG data to determine the vital health signs prediction.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A smart health monitoring system, comprising:
an attachable device 100 configured to be attached on a targeted area of an aquatic animal, wherein the attachable device 100 comprising,
an imaging device 101 configured to initially scan the targeted area,
a photoplethysmography (PPG) device 102 configured to generate a PPG data from a plurality of sensors,
a microcontroller 108 programmed to collect, process, and classify the data received from the imaging device 101 and the PPG device 102,
a display screen 109 configured to live display a processed data received from the microcontroller 108,
a wireless module 110 configured to transmit the processed data wirelessly to an external processing device 200, and
a power module 111 configured to supply power to the microcontroller 108;
the external processing device 200 configured for machine learning based processing to infer the vital health signals in real time by implementing a machine learning model.

2. The smart health monitoring system as claimed in claim 1, wherein the plurality of sensors comprising a temperature sensor 103, a blood pressure sensor 104, an oxygen saturation (SpO2) sensor 105, a pulse sensor 106, and a respiration sensor 107.

3. The smart health monitoring system as claimed in claim 1, wherein the power module 111 comprising a charging port 112, a battery 113, and a power management circuit 114, wherein the battery 113 is connected to the power management circuit 114 which feeds power to the microcontroller 108, and the battery 113 is charged via the charging port 112 which receives the power when connected to an external charging station.

4. The smart health monitoring system as claimed in claim 1, wherein the external processing device 200 reconstructs a detailed 4-dimensional micro blood vessels image based on a processed scan data by the imaging device 101, which is further fused with a processed PPG data generated by the PPG device 102, in order to generate a specific baseline profile of the vital health signal data for each aquatic animal under measurement.

5. The smart health monitoring system as claimed in claim 4, wherein the external processing device 200 generates the specific baseline profile of the vital health signal data for each aquatic animal under measurement, using a data visualization platform.

6. The smart health monitoring system as claimed in claim 1, wherein the external processing device 200 implements a machine learning model to identify key features in real-time including, but not limited to systolic blood pressure (SBP), diastolic blood pressure (DBP), SpO2, and respiration rate.

7. The smart health monitoring system as claimed in claim 1, wherein the external processing device 200 can be selected from, but not limited to a personal computer, a laptop, a tablet, a smartphone, a mobile phone, and a personal digital assistance.

8. The smart health monitoring system as claimed in claim 1, wherein the wireless module 111 transmits the processed scan data and PPG data to the external processing device 200 via a wireless network selected from, but not limited to Wi-Fi, Bluetooth™, ZigBee™, Cellular, and Satellite.

9. A smart health monitoring method, comprising the steps of:
concurrently recording an initial scan data and an initial PPG data measured via an imagine device 101 and a PPG device 102, respectively, wherein the recording of the data lasts for at least 5 minutes;
storing the recorded data on an external processing device 200;
tracking a live PPG data from the PPG device 102 that is still attached to an aquatic animal;
analyzing the recorded data to determine temporal correlations between the recorded data and the live PPG data, wherein the temporal correlations correlate the recorded data to the live PPG data by running a Deep Neural Network (DNN) model to identify key features including, but not limited to systolic blood pressure (SBP), diastolic blood pressure (DBP), SpO2, and respiration rate;
extracting a more detailed vital health signs from the recorded data to reconstruct aquatic animal's hemodynamics system by running a Recurrent Neural Network (RNN) model;
storing a historical record of the medical data of the aquatic animal on a memory device of an attachable device 100;
wirelessly transmitting the historical record of the medical data of the aquatic animal to the external processing device 200;
receiving by the external processing device 200, one or more medical threshold values from the PPG device 102 and/or a plurality of sensors 103, 104, 105, 106, 107;
transmitting an alert to the external processing device 200 in response to the data in the historical records exceeding one or more of the medical threshold values.
